(19) United States
(12) Patent Application Publication
(10) Pub. No.: US 2014/0319238 A1
(43) Pub. Date: Oct. 30, 2014

(54) PERFUME SPRAYER

(71) Applicant: CHIEN CHANG SU, New Taipei City (TW)

(72) Inventor: CHIEN CHANG SU, New Taipei City (TW)

(21) Appl. No.: 13/870,462

(22) Filed: Apr. 25, 2013

(51) Int. Cl. A45D 34/00

(52) U.S. Cl. CPC A45D 34/00; A45D 2034/002; A45D 2034/005
USPC 239/70; 239/306

(57) ABSTRACT

A perfume sprayer comprising a body, a first transmission unit, a second transmission unit and a control circuit is disclosed. The body has a bracket disposed inside the body for receiving a plurality of bottles, and the bottles have releasing units and contain distinct perfume. The first transmission unit is engaged to the bracket. The second transmission unit is disposed at beneath of the bracket, wherein one end of the driving element pivotally connects to a pushing element. The control circuit is coupled to the first transmission unit and the second transmission unit for controlling the bracket rotation in a predetermined time, to achieve function of multi perfumes spray respectively.

### BACKGROUND OF THE INVENTION

1. Field of the Invention

The present invention relates to a perfume sprayer, particularly to a perfume sprayer achieving fragrant air purpose by spraying different kinds of perfumes in different time.

2. Description of Related Art

In home living or public place, a perfume sprayer is normally used, for diminish the smell of the air and then achieve the fragrance, by means of spraying the perfume. The conventional sprayer releases the perfume is either automatically discharged in a predetermined time or manually ejected by means of our hand; these ways are unitary and boring because that only one perfume is utilized. Unitary perfume always lacks of diversification, and may not attract someone else's attention in the long run. In order to resolve the unitary problem of the perfume sprayer, some new configuration is achieved and invented which will be addressed and disclosed, to have function of multi perfumes spray.

### SUMMARY OF THE INVENTION

The primary object of the present invention is to have a perfume sprayer with function of multi perfumes spray in different time, so as to have fragrant air or environment.

To achieve the above purpose, a perfume sprayer is disclosed. The perfume sprayer comprises a body, a first transmission unit, a second transmission unit and a control circuit. The body has a bracket disposed inside the body for receiving a plurality of bottles, and the bottles have releasing units and contain distinct perfume, and the bracket has a plurality of openings corresponding to the bottles respectively. The first transmission unit is engaged to the bottom of the bracket. The second transmission unit is disposed at beneath of the bracket and has an eccentric wheel abutting a driving element, wherein one end of the driving element pivotally connects to a pushing element, and the pushing element is corresponded to the opening of the bracket. The control circuit is coupled to the first transmission unit and the second transmission unit, wherein the first transmission unit is controlled to drive the bracket rotating an angle in a predetermined time by the control circuit, and to make distinct bottles respectively correspond to the pushing element of the second transmission unit; wherein the second transmission unit drives the pushing element to propel the bottle upward, to respectively squeeze the releasing units and then release the perfumes of the bottles.

According to one embodiment of the perfume sprayer, wherein the first transmission unit is composed of motor and gear set, and the bottom of the bracket has a gear engaged to the first transmission unit, to have the bracket rotatable by means of the first transmission unit.

According to one embodiment of the perfume sprayer, wherein another end of the driving element is connected to a spring, and another end of the spring is fixed to the body; wherein the pushing element is moved downward to located at beneath of the opening by the tensile force of the spring if the eccentric wheel returns.

According to one embodiment of the perfume sprayer, wherein the control circuit is coupled to a plurality of LEDs and optional buttons, and the LEDs and the optional buttons are exposed and disposed at surface of the body for setting the interval of spray or option of perfume.

According to one embodiment of the perfume sprayer, wherein the control circuit is coupled to a LCD monitor, and the LCD monitor is exposed and disposed at surface of the body for revealing the time.

In short, the perfume sprayer of the present invention may have function of multi perfumes spray in different time, so as to have fragrant air or environment. Therefore, unitary and boring circumstance will be prevented, and lack of diversification is not happened any more. In the long run, it may easily attract someone else's attention to make the invention applicable.

To further understand the techniques, means and effects of the instant disclosure applied for achieving the prescribed objectives, the following detailed descriptions and appended drawings are hereby referred, such that, through which, the purposes, features and aspects of the instant disclosure can be thoroughly and concretely appreciated. However, the appended drawings are provided solely for reference and illustration, without any intention to limit the instant disclosure.

### BRIEF DESCRIPTION OF THE DRAWINGS

FIG. 1 shows the schematic diagram of the present invention;

FIG. 2 shows the structural diagram of the present invention;

FIG. 3 shows another structural diagram of the present invention;

FIG. 4 shows the exchanged action of the bottle according to the present invention;

FIG. 5 shows the spray action of the present invention.

### DETAILED DESCRIPTION OF THE PREFERRED EMBODIMENTS

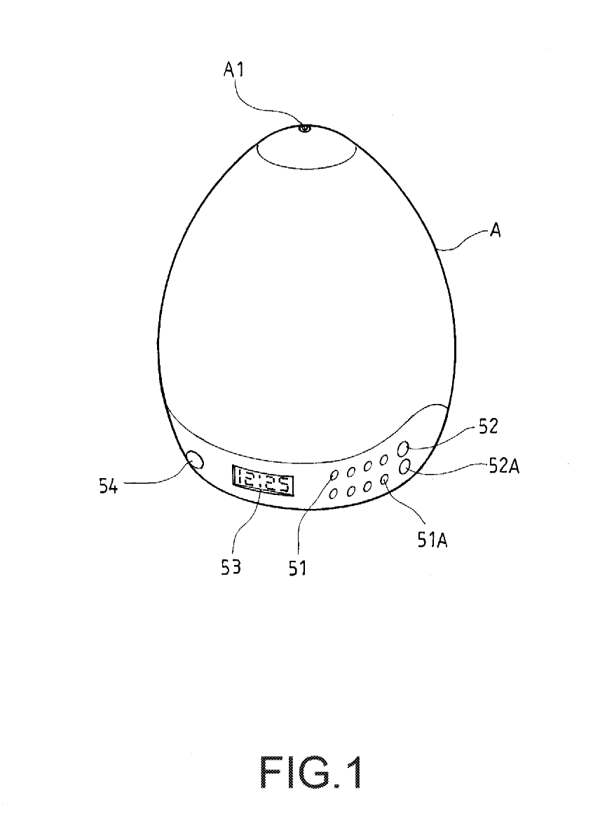

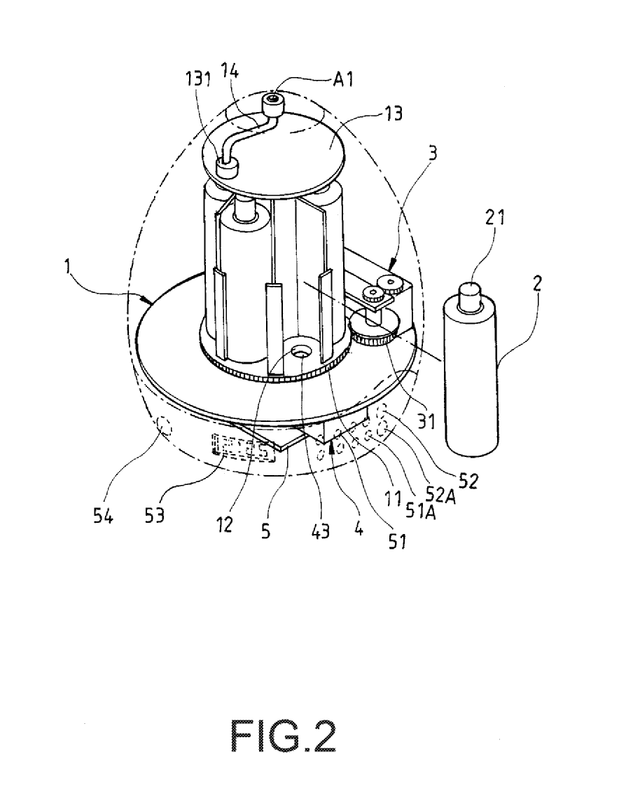

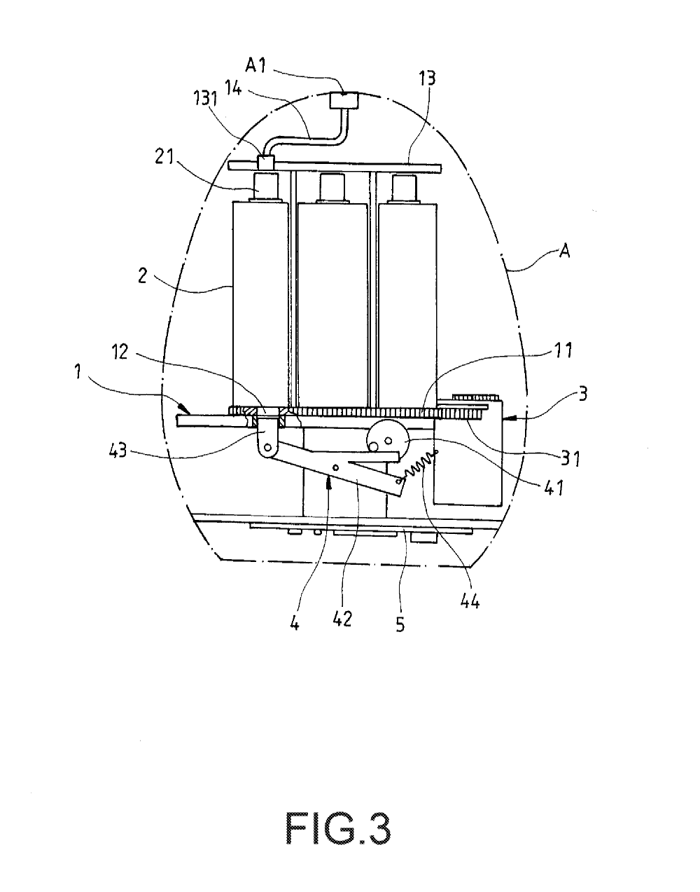

Referring to the FIG. 1, FIG. 2 as well as FIG. 3 concurrently; FIG. 1 shows the schematic diagram of the present invention; FIG. 2 shows the structural diagram of the present invention; FIG. 3 shows another structural diagram of the present invention. As demonstrated, the perfume sprayer has a body A, an outlet A1 disposed on the body A, and a bracket 1 disposed inside the body A. The bracket 1 is disposed inside the body A for receiving a plurality of bottles 2, in which each bottle 2 has a releasing unit 21 and contains distinct perfume respectively. The beneath of the bracket 1 has a gear 11 engaged to a first transmission unit 3, in which the first transmission unit 3 is composed of motor and gear set 31. The lower side of the bracket 1 has a plurality of openings 12 corresponding to each bottle 2 respectively; the upper side of the bracket 1 has a retainer 13, and the retainer 13 contains a discharger 131 connecting to a outlet A1 by means of the pipe 14.

The bracket 1 further has a second transmission unit 4 disposed at the beneath of the bracket 1. The second transmission unit 4 has motor and an eccentric wheel 41 abutting on a driving element 42. In the embodiment, one end of the driving element 42 is pivotally connects to a pushing element 43, and the pushing element 43 is corresponded to the opening 12 of the bracket 1. Furthermore, another end of the driving element 42 is connected to a spring 44, and another end of the spring 44 is fixed to the body A. A control circuit 5 is coupled to the first transmission unit 3, the second transmission unit 4, a plurality of LEDs 51, 51A, a plurality of optional buttons 52, 52A and a LCD monitor 53 for revealing time. The LEDs 51, 51A, the optional buttons 52, 52A and the LCD monitor 53 are exposed and disposed at surface of the body A for setting the interval of spray or option of perfume by means of the optional buttons 52, 52A. Meanwhile, the setting results may be displayed in the LEDs 51, 51A. Besides, the entire power supply may be turned on/off by the switch 54.

By means of aforementioned configuration, the perfume sprayer may have function, for example, of selecting particular perfume through the optional button 52, and revealing the option in the LED 51. Besides, the perfume spraying interval (e.g. every 5, 10, 30, or 60 minutes) may also be regulated and controlled by means of the optional button 52A, and then demonstrated in the LED 51A.

The first transmission unit 3 may be controlled to drive the bracket 1 rotating an angle in a predetermined time by means of the control circuit 5, and therefore to make distinct bottles 2 respectively correspond to the pushing element 43 of the second transmission unit 4. Thus the second transmission unit 4 may drive the pushing element 43 to propel the bottles 2 upward, to respectively squeeze the releasing units 21 and then release the perfumes from the bottles 2. Preferably, distinct perfumes may be respectively squeezed to release in different time, through the discharger 131, the pipe 14 and the outlet A1 in turn. In this manner, different perfumes may be respectively released to the air so as to achieve fragrant function.

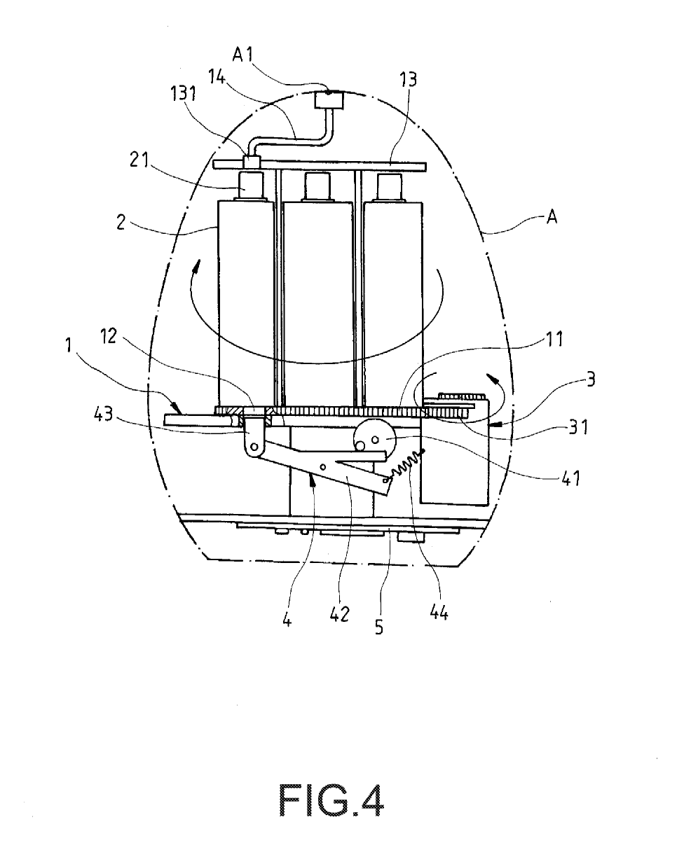

Referring to the FIG. 4 as well as FIG. 1 and FIG. 2, FIG. 4 shows the exchanged action of the bottle according to the present invention. When the perfume sprayer is used, the bottles 2 may contains distinct perfumes, e.g. rose or orchid, and then be located in the bracket 1. By means of setting the optional buttons 52, 52A, the control circuit 5 may control the rotation of the bracket 1 and spraying time of the perfume, therefore the selection of the perfume used and the spray interval are hence controlled and regulated. Further, the LEDs 51, 51A may reveal and demonstrate the setting results. Preferably, the spraying time of the perfume can be once a day, or morning and afternoon once respectively, for example. The first transmission unit 3 is controlled to drive the gear 11 and then urge the bracket 1 rotating an angle in a predetermined time by the setting of the control circuit 5; therefore distinct bottles 2 can be respectively corresponded to the pushing element 43 of the second transmission unit 4, so that the pushing element 43 may be moved and then propel the bottle 2 moving upward.

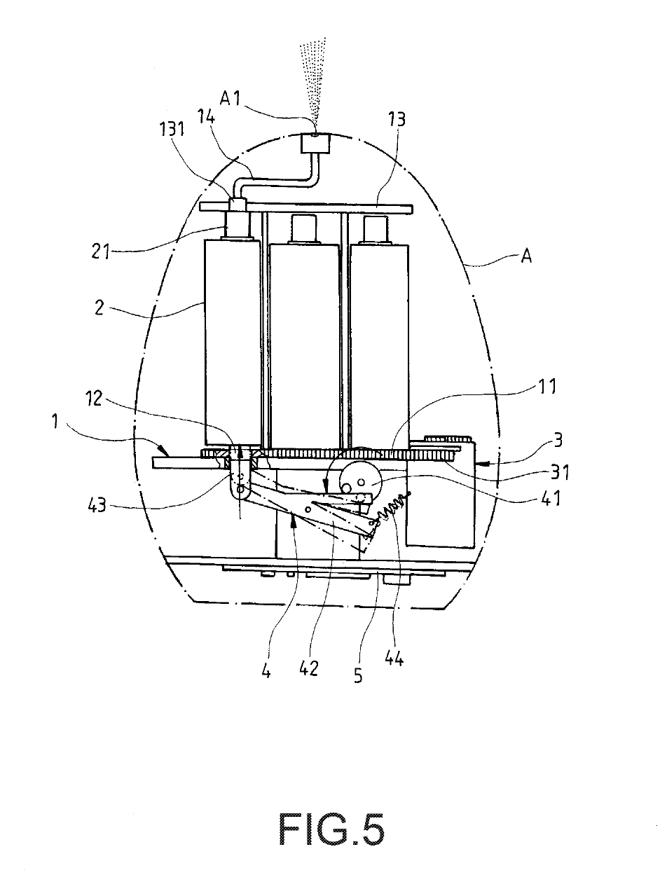

Please referring to the FIG. 5 as well as FIG. 1 and FIG. 2, FIG. 5 shows the spray action of the present invention. More descriptively, the control circuit 5 drives the first transmission unit 3 and the gear 11, to urge the bracket 1 to rotate in the predetermined time, different bottles 2 will be corresponded to the pushing element 43 of the second transmission unit 4. Meanwhile, the control circuit 5 may drive the second transmission unit 4 and make the eccentric wheel 43 repeatedly rotate. In this manner, the driving element 42 may be urged downward, and then the end of the driving element 42 approaching to the pushing element 43 may be upward, so that the pushing element 43 with pivotally connection to the driving element 42 is hence moved upward. Afterward, the pushing element 43 is forced to pass through the opening 12 and then press the bottle 2, to have the releasing unit 21 squeezed; therefore the perfume stored in distinct bottles 2 may be respectively (i.e. in different time) discharged, through the discharger 131, the pipe 14 and the outlet A1 of the body A in turn. The function of air fragrance by different perfumes is achieved. Besides, the selection of spraying perfumes may be displayed by LED 51 on the outer surface of the body A. When the eccentric wheel 41 is returned, the pushing element 43 may be moved downward to locate at beneath of the opening 12 by the tensile force of the spring 44, since the spring 44 is connected to opposite end of the driving element 42. In this manner, the perfume sprayer may be waited for next spray action. In some other embodiment, selection from the setting of the optional buttons 52, 52A and display from the LEDs 51, 51A, for option of different perfume, may also be available.

Summarily, the perfume sprayer may rotate bracket 1 and drive pushing element 43, to squeeze the perfume bottles 2 and then release the different perfumes inside the bottles 2 respectively (i.e. in different time). Therefore, the perfume sprayer of the present invention may have function of multi perfumes spray in different time, so as to achieve fragrant air or environment. In this manner, unitary and boring circumstance will be prevented, and lack of diversification is not happened any more. In the long run, it may easily attract someone else's attention to make the invention applicable.

The above-mentioned descriptions merely represent the preferred embodiments of the instant disclosure, without any intention or ability to limit the scope of the instant disclosure which is fully described only within the following claims. Various equivalent changes, alterations or modifications based on the claims of instant disclosure are all, consequently, viewed as being embraced by the scope of the instant disclosure.

What is claimed is:

1. A perfume sprayer comprising:
a body having a bracket disposed inside the body for receiving a plurality of bottles, wherein the bottles have releasing units and contain distinct perfume, and the bracket has a plurality of openings corresponding to the bottles respectively;
a first transmission unit engaging to the beneath of the bracket;
a second transmission unit, disposed at beneath of the bracket and having an eccentric wheel abutting on a driving element, wherein one end of the driving element pivotally connects to a pushing element, and the pushing element is corresponded to the opening of the bracket; and
a control circuit coupled to the first transmission unit and the second transmission unit, wherein the first transmission unit is controlled to drive the bracket rotating an angle in a predetermined time by the control circuit, and to make distinct bottles respectively correspond to the pushing element of the second transmission unit; wherein the second transmission unit drives the pushing element to propel the bottle upward, to respectively squeeze the releasing units and then release the perfumes of the bottles.

2. The perfume sprayer as defined in claim 1, wherein the first transmission unit is composed of motor and gear set, and the beneath of the bracket has a gear engaged to the first transmission unit, to have the bracket rotatable by means of the first transmission unit.

3. The perfume sprayer as defined in claim 1, wherein another end of the driving element is connected to a spring, and another end of the spring is fixed to the body; wherein the pushing element is moved downward to located at beneath of the opening by the tensile force of the spring if the eccentric wheel returns.

4. The perfume sprayer as defined in claim 1, wherein the control circuit is coupled to a plurality of LEDs and optional buttons, and the LEDs and the optional buttons are exposed and disposed at surface of the body for setting the interval of spray or option of perfume.

5. The perfume sprayer as defined in the claim 1, wherein the control circuit is coupled to a LCD monitor, and the LCD monitor is exposed and disposed at surface of the body for revealing the time.
